(19) United States
(12) Patent Application Publication
(10) Pub. No.: US 2003/0082562 A1
(43) Pub. Date: May 1, 2003

(54) Maxam gilbert g/a sequence analysis by DHPLC

(76) Inventors: David P. Hornby, Cheshire (GB); Mark Dickman, Sheffield (GB)

Correspondence Address: CHRISTOPHER M. HOLMAN, TRANSGENOMIC, INC., 2032 CONCOURSE DRIVE, SAN JOSE, CA, 95131, US

(21) Appl. No.: 10/058,151

(22) Filed: Jan. 25, 2002

Related U.S. Application Data
(63) Continuation of application No. 09/718,826, filed on Nov. 22, 2000, now abandoned.

(51) Int. Cl. C12Q 1/68; C07H 21/04

(52) U.S. Cl. 435/6; 536/25.4; 210/656

(57) ABSTRACT

The present invention describes a method for generating a chromatographic DNA sequencing ladder useful for phasing a chromatographic separation of fragments derived from the DNA sequence, e.g., a DNA footprinting reaction. The chromatographic separation is preferably accomplished using ion pairing reverse phase high performance liquid chromatography under conditions that are substantially free of multivalent cations that are free to interfere with polynucleotide separations.

### FIELD OF THE INVENTION

The present invention is directed to methods and materials useful for characterizing a separation of DNA fragments by high performance liquid chromatography. In one aspect, the invention can be used to phase chromatographic peaks generated by separating DNA fragments of varying lengths sharing a common 5′-end.

### BACKGROUND OF THE INVENTION

Traditional DNA sequencing techniques are based on electrophoretic procedures using high-resolution denaturing polyacrylamide gels, and more recently capillaries. These so-called “sequencing” gels and capillaries are capable of resolving single-stranded oligonucleotides up to 800 bases in length which differ is size by a single deoxynucleotide. In practice, for a given region to be sequenced, a set of labeled, single-stranded oligonucleotides is generated, the members of which have one fixed end and which differ at the other end by each successive deoxynucleotide in the sequence. The key to determining the sequence of deoxynucleotides is to generate, in four separate enzymatic or chemical reactions, all oligonucleotides that terminate at the variable end in adenine (A), thymine (T), cytosine (C) or guanine (G). The oligonucleotide products of the four reactions are then resolved on adjacent lanes of a sequencing gel. Because all possible oligodeoxynucleotides are represented among the four lanes, the DNA sequence can be read directly from the four “ladders” of oligonucleotides.

The two methods that have traditionally been used to determine DNA sequences, the enzymatic dideoxy method and the chemical method, differ primarily in the technique used to generate the ladder of oligonucleotides. In the dideoxy method, originally developed by Sanger and co-workers, a DNA polymerase is used to synthesize a copy of a single-stranded DNA template by extension of a primer that is complementary to the template (Sanger et al. (1977) Proc. Natl. Acad. Sci. USA 74:5463-67). The method exploits the ability of certain DNA polymerases to use 2′,3′-dideoxynucleoside triphosphates (ddNTPs) as substrates. When a ddNMP is incorporated at the 3′ end of the growing primer chain, chain elongation is terminated at G, A, T or C because the primer chain now lacks a 3′-hydroxyl group. To generate the four sequencing ladders, only one of the four possible ddNTPs is included in each of the four reactions. The ddNTP:dNTP ratio in each reaction is adjusted such that a portion of the elongating primer chains terminates at each occurrence of the base in the template DNA corresponding to the included complementary ddNTP. In this way, each of the four elongation reactions contains a population of extended primer chains, all of which have a fixed 5′ end determined by the annealed primer and a variable 3′ end terminating at a specific dideoxynucleotide.

In the chemical method of DNA sequencing, also referred to as the Maxam-Gilbert method, the four sets of deoxyoligonucleotides are generated by subjecting a purified 3′ or 5′-end-labeled deoxyoligonucleotides to a base-specific chemical reagent that randomly cleaves DNA at one or two specific nucleotides (Maxam & Gilbert (1977) Proc. Natl. Acad. Sci. USA 74:560-64). Because only end-labeled fragments are observed following autoradiography of the sequencing gel, four DNA ladders are generated. This method is based on the ability of hydrazine, dimethyl sulfate (DMS), or formic acid to specifically modify bases within the DNA molecule. Piperdine is then added to catalyze strand breakages at these modified nucleotides. The specificity resides in the first reaction with hydrazine, DMS or formic acid, which react with only a few percent of the bases. The second reaction, piperdine strand cleavage, must be quantitative.

Because of its relative ease and efficiency, the dideoxy method is more commonly used than the chemical cleavage method for routine sequencing. The major disadvantage of dideoxy sequencing is that the composition or secondary structure of the template can sometimes cause premature termination by DNA polymerase. DNA is sometimes encountered that cannot be accurately sequenced by the dideoxy method. A major advantage of the chemical method is that problems associated with premature DNA polymerase termination are eliminated, which permits sequencing of stretches of DNA that cannot be sequenced by the enzymatic method. In addition, obtaining the sequence of shorter regions of DNA using the chemical method does not require amplification in vivo or in vitro, such as is required for dideoxy sequencing. Finally, chemical cleavage is the only sequencing method available for small oligonucleotides.

DNA sequencing is used not only determine DNA sequences de novo, but is often applied to a DNA molecule of known sequence. An important example is the use of DNA sequencing methodology to generate a series of size-differentiated labeled oligonucloetide standards for establishing the identity of oligonucleotide fragments generated in a DNA analysis technique, e.g., DNA footprinting. The sequencing reaction is typically run on a sequencing gel in parallel with a sample of the same DNA that has been treated in some manner to yield cleavage products. All of the oligonucleotides generated in both reactions are normally radio-labeled at the 5′-end, so that detected bands share a common 5′-end. Thus, the length of each oligonucleotide is a function of location of the 3′-end, which depends upon where the original DNA fragment was cleaved in the sequencing or other cleavage reaction. Oligonucleotides sharing a common 3′-end will form bands that migrate the same distance on a sequencing gel. Thus, the 3′-end of any band can be determined if the identity of a co-migrating DNA sequencing band is known from previous sequence determination. This process of orienting the locations of DNA cleavages by alignment with bands generated by a sequencing reaction of a known sequence is sometimes referred to as “phasing.”

Normally when using DNA sequencing to phase a DNA analysis electrophoresis gel it is not necessary to run all four sequencing reactions, since even one reaction is usually sufficient to orient oneself in a known sequence. For example, Tullius et al. describe phasing a DNA footprinting reaction with a single Maxam-Gilbert reaction, i.e, the G-specific reaction, as described supra.

DNA “footprinting,” a procedure used to localize a protein binding site on a DNA molecule (Galas & Schmitz (1978) Nucleic Acids Res. 5:3157). DNA footprinting can be used, for example, to identify a site where a transcription factor binds. The technique entails allowing a protein of interest to bind to a labeled DNA molecule containing a sequence that is recognized by the protein. The DNA-protein complex is digested by a nuclease, normally deoxyribonuclease I (Dnase I), or chemically using, e.g., hydroxyl radicals generated by Fe(EDTA)² (Tullius and Dombroski (1986) Proc. Natl. Acad. Sci. USA 83:5469). The chemistry of hydroxyl radical induced polynucleotide cleavage is described, for example, in Balasubramanian et al., (1998) Proc. Natl. Acad. Sci. USA 95:9738-43, incorporated by reference herein in its entirety. Regions of the DNA molecule covered by the bound protein are protected from digestion while the rest of the DNA backbone is cut normally. If the products of this reaction are separated on an electrophoresis gel, e.g., such as is used for DNA sequencing, the amount of digestion at each position in the sequence can be seen. A blank region of the autoradiograph of the gel, called a “footprint,” is found at a location corresponding to the sequence where the protein binds specifically to the DNA.

While DNA analysis has traditionally relied heavily on the use of radioactively-labeled DNA and gel electrophoresis, there are substantial disadvantages associated with their use. The use of radioactivity requires that special safety precautions be taken, and the disposal of the radioactive waste that necessarily results from these methods can be inconvenient and expensive. Moreover, the use of sequencing gels is messy, time consuming, and can yield inconsistent results in the hands of different technicians. Gel banding patterns are also notoriously difficult to quantify and interpret. Sequencing gels produced in different laboratories are often difficult to compare quantitatively due to the reproducibility problems inherent to pouring and running gels. The bands representing distinct polynucleotide populations are often curved rather than straight, their mobility and shape can change across the width of the gel, and lanes and bands can mix with each other. These inaccuracies typically stem from the lack of uniformity and homogeneity of the gel bed, electroendosmosis, thermal gradient and diffusion effects, as well as host of other factors. Inaccuracies of this sort can lead to serious distortions and inaccuracies in the display of the separation results. In addition, the band display data obtained from gel electrophoresis separations is not quantitative dr accurate because of the uncertainties related to the shape and integrity of the bands. True quantitation of linear band array displays produced by gel electrophoresis separations cannot be achieved, even when the linear band arrays are scanned with a detector and the resulting data is integrated, because the linear band arrays are scanned only across the center of the bands. Since the detector only sees a small portion of any given band and the bands are not uniform, the results produced by the scanning method are not accurate and can even be misleading. Furthermore, methods for visualizing gel electrophoretic separations, such as staining or autoradiography, tend to be cumbersome and time consuming. Furthermore, gel electrophoresis is difficult to automate and to practice in a high-throughput manner. These limitations inherent in the use of gel electrophoresis have been associated with false positives in assays and poor qualitative analysis.

Furthermore, it is often difficult to achieve high resolution separations for very small DNA fragments using gel electrophoresis. This poses a significant limitation when attempting to analyze DNA cleavage products in cases where the cleavage occurs near the labeled end, thereby restricting the applicability of gel electrophoresis in certain DNA sequencing applications, or in other applications that involve the separation of small fragments of DNA.

As an alternative to radiolabeling and gel electrophoresis, non-radiolabeled DNA can be analyzed using high performance liquid chromatography (HPLC). In particular, ion pairing reverse phase HPLC (IP-RP-HPLC) is form of HPLC that has been shown to be effective at separating DNA molecules in some instances. IP-RP-HPLC is characterized by the use of a reverse phase (i.e., hydrophobic) stationary phase and a mobile phase that includes an alkylated cation (e.g., triethylammonium) that is believed to form a bridging interaction between the negatively charged DNA and non-polar stationary phase. The alkylated cation-mediated interaction of DNA and stationary phase can be modulated by the polarity of the mobile phase, conveniently adjusted by means of a solvent that is less polar than water, e.g., acetonitrile. Performance is enhanced by the use of a non-porous separation medium, as described in U.S. patent application No. 5,585,236. However, before IP-RP-HPLC can be successfully applied to DNA analysis techniques such as DNA footprinting, a means for phasing an HPLC-based DNA separation method must be made available. The instant invention satisfies this need, and thus represents a valuable and timely contribution to various fields of scientific endeavor including, molecular biology, genetics and medicine.

### SUMMARY OF THE INVENTION

The present invention provides novel methods and reagents for phasing an HPLC-based separation of polynucleotide fragments, especially DNA fragments. In one aspect, the instant invention provides a chromatographic DNA sequencing ladder useful for phasing a chromatographic separation of fragments derived from the DNA sequence. For example, the DNA fragments can be derived by treatment of the DNA under conditions that can result in the cleavage of some of the phosphodiester bonds linking the nucleotides of the DNA molecule, e.g., a DNA footprinting reaction.

One aspect of the invention is a method for identifying the 3′-end of a polynucleotide appearing as a chromatographic peak in a first IP-RP-HPLC chromatogram, wherein said polynucleotide has been generated by subjecting a precursor nucleic acid of known sequence to a first cleavage reaction, and wherein said polynucleotide shares with said precursor nucleic acid a common 5′-end. The method comprises the steps of providing a plurality of polynucleotides, wherein said plurality of polynucleotides have been generated by subjecting the precursor nucleic acid of known sequence to a second cleavage reaction, wherein said second cleavage reaction is base-discriminating and is distinct from the first cleavage reaction, and wherein said plurality of polynucleotides share with said precursor nucleic acid and with each other a common 5′-end; separating said plurality of polynucleotides by IP-RP-HPLC, wherein the IP-RP-HPLC separation conditions are substantially the same as those used to generate said first IP-RP-HPLC chromatogram; detecting said plurality of polynucleotides as they elute from the IP-RP-HPLC separation, thereby generating a second HPLC chromatogram, wherein the 3′-end of a polynucleotide appearing as a chromatographic peak in said second IP-RP-HPLC chromatogram can be determined based on the known sequence of the precursor nucleic acid; and comparing said first IP-RP-HPLC chromatogram with said second IP-RP-HPLC chromatogram, wherein the 3′-end of a polynucleotide appearing as a chromatographic peak in said first IP-RP-HPLC chromatogram can be identified based on its elution position relative to a peak appearing in said second IP-RP-HPLC chromatogram whose 3′-end is known.

In a preferred embodiment of the invention the IP-RP-HPLC employs a separation medium that is substantially free of multivalent cations that are capable of interfering with polynucleotide separations.

In another preferred embodiment of the invention, the polynucleotide is DNA.

In an aspect of the invention, the separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein said surfaces are non-polar.

In another aspect of the invention, the separation medium comprises polymer beads having an average diameter of 0.5 to 100 microns, said beads being unsubstituted polymer beads or polymer beads substituted with a moiety selected from the group consisting of hydrocarbon having from one to 1,000,000 carbons.

In yet another aspect of the invention the separation medium comprises a monolith.

Preferred embodiments of the invention employ a separation medium that has been subjected to acid wash treatment to remove any residual surface metal contaminants and/or has been subjected to treatment with a multivalent cation binding agent.

In one aspect of the invention, the IP-RP-HPLC employs a mobile phase comprising a solvent selected from the group consisting of alcohol, nitrite, dimethylformamide, tetrahydrofuran, ester, ether, and mixtures of one or more thereof, preferably acetonitrile.

In yet another aspect of the invention, said mobile phase comprises a counterion agent selected from the group consisting of lower alkyl primary amine, lower alkyl secondary amine, lower alkyl tertiary amine, lower trialkylammonium salt, quaternary ammonium salt, and mixtures of one or more thereof.

In a preferred embodiment of the invention, the counterion agent is selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyidiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof. Tetrabutylammonium acetate and triethylammonium acetate are particularly preferred counterion agent.

In preferred embodiments of the invention, the counterion agent includes an anion, said anion is selected from the group comprising acetate, carbonate, phosphate, sulfate, nitrate, propionate, formate, chloride, and bromide.

In particularly preferred embodiments of the invention, the detection is achieved using Matched Ion Polynucleotide Chromatography. In one aspect the DNA molecule is detectably labeled, preferably by means of a fluorescent label. In a preferred embodiment of the invention the label is selected from the group consisting of FAM, JOE, TAMRA, ROX, HEX, TET, Cy3, and Cy5.

In a preferred embodiment of the invention the method is used to identify the 3′-ends of a plurality of polynucleotides appearing as chromatographic peaks in said first IP-RP-HPLC chromatogram by comparing said first IP-RP-HPLC chromatogram and said second IP-RP-HPLC chromatogram.

In another preferred embodiment of the invention, the first cleavage reaction involves the use of a DNA cleavage reagent that cleaves DNA that is not protected by a bound protein.

In a preferred embodiment of the invention the bound protein is a mismatch binding protein, especially a protein selected from the group consisting of T4 endonuclease VII, T7 endonuclease I, S1 nuclease, mung bean endonuclease, MutY protein, MutS protein, MutH protein, MutL protein, cleavase, and CELI. In a particularly preferred embodiment of the invention, the mismatch binding protein is CELI.

In another preferred embodiment of the invention the bound protein is a protein involved in DNA transcription, replication, and recombination

In still another preferred embodiment of the invention the bound protein is a protein selected from the group consisting of transcription factors, enhancers, repressors, and histones.

In yet another preferred embodiment of the invention, the bound protein is a protein that binds to a Holliday junction, especially RuvA.

In another preferred embodiment of the invention, the cleavage reagent is a hydroxyl radical.

In another embodiment of the invention, the cleavage reagent is a nuclease, especially DNase I.

In one aspect of the invention, said second cleavage reaction is a reaction that results in the specific cleavage of a DNA sequence preferentially after one, two or three of the bases selected from the group consisting of adenine, guanine, cytosine, and thymine. In a preferred embodiment, said second cleavage reaction is a chemical cleavage DNA sequencing reaction. In a particularly preferred embodiment, said sequencing reaction is an A+G specific DNA sequencing reaction, especially a DNA sequencing reaction that is based on partial acidic hydrolyses of DNA in the presence of diphenylamine and proceeds via depurination/5′,3′-elimination.

### BRIEF DESCRIPTION OF THE FIGURES

FIG. 1a is a chromatogram representing the IP-RP-HPLC separation of the FAM-labeled HJ3 strand cleaved by hydroxyl radical treatment in the absence of DNA binding protein, as described in Example 1.

FIG. 1b is a chromatogram representing the IP-RP-HPLC separation of the FAM-labeled HJ3 strand cleaved by hydroxyl radical treatment in the presence of the DNA binding protein RuvA, as described in Example 1.

FIG. 1c is a chromatogram representing the IP-RP-HPLC separation of the FAM-labeled HJ3 strand cleaved by a G+A Maxam-Gilbert sequencing reaction, generated to phase the DNA footprinting chromatograms of FIGS. 1a and 1b, as described in Example 1.

FIG. 2a is a chromatogram representing the IP-RP-HPLC separation of the TET-labeled HJ4 strand cleaved by hydroxyl radical treatment in the absence of DNA binding protein, as described in Example 1.

FIG. 2b is a chromatogram representing the IP-RP-HPLC separation of the TET-labeled HJ4 strand cleaved by hydroxyl radical treatment in the presence of the DNA binding protein RuvA, as described in Example 1.

FIG. 3 is a chromatogram representing the IP-RP-HPLC separation of a FAM-labeled synthetic Holliday junction oligonucleotide (SEQ ID NO: 5) cleaved by a G+A Maxam-Gilbert sequencing reaction, as described in Example 2.

FIG. 4 is a chromatogram representing the IP-RP-HPLC separation of the HEX-labeled oligonucleotide TATA2 (SEQ ID NO: 6) cleaved by a G+A Maxam-Gilbert sequencing reaction, as described in Example 2.

### DETAILED DESCRIPTION OF THE INVENTION

As described above, the need exists for a method for phasing an HPLC-based separation of DNA fragments. The present invention provides novel methods and reagents that satisfy this need. In particular, the instant invention provides a chromatographic DNA sequencing ladder useful for phasing a chromatographic separation of fragments derived from the DNA sequence. For example, the DNA fragments can be derived by treatment of the DNA under conditions that can result in the cleavage of some of the phosphodiester bonds linking the nucleotides of the DNA molecule, e.g., a DNA footprinting reaction. It is therefore an object of the instant invention to provide improved methods and reagents for phasing an HPLC-based DNA separation.

Practice of the instant invention can entail a variety of techniques and methods known to one of skill in the art. Such methods are widely available and provided, for example, in Molecular Cloning: a Laboratory Manual: 2nd edition, 3 Volumes, Sambrook et al, 1989, Cold Spring Harbor Laboratory Press (or later editions of the same work) or Current Protocols in Molecular Biology, Second Edition, Ausubel et al. eds., John Wiley & Sons, 1992.

The instant invention provides a DNA sequencing ladder that can be used to phase an HPLC-based DNA separation. In one aspect, the separated DNA consists of a series of DNA fragments generated by cleavage of a common precursor polynucleotide, where the fragments share a common 5′-end but a differ in length based upon the identity of the 3′-end, i.e., the location of cleavage. In a preferred embodiment of the invention, the series of DNA fragments is the result of a DNA footprinting experiment. Such an experiment can be used in conjunction with the instant invention to identify the site on a DNA sequence where a protein of interest binds, particularly in a sequence-specific manner. In a preferred embodiment, the protein is one that binds to DNA in a sequence-specific manner. Examples of such proteins include transcription factors, enhancers, repressors, and a variety of proteins involved in DNA transcription, replication, and recombination. In a particularly preferred embodiment of the invention DNA binding protein binds specifically to a Holliday junction (an intermediate in prokaryotic homologous recombination), e.g., RuvA (Aniyoshi et al. (2000) Proc. Natl. Acad. Sci. U S A 97:8257-62).

In a particularly preferred embodiment of the invention, the binding protein is a protein capable of binding to a mutation or base mismatch, e.g., T4 endonuclease 7, T7 endonuclease 1, S1 nuclease, mung bean endonuclease, MutY protein, MutS protein, MutH protein, MutL protein, cleavase, and CELI. These and other base mismatch recognition enzymes, and their use in the detection of mutations and other polymorphisms are discussed in U.S. Pat. Nos. 5,459,039; 6,027,898; and 5,869,245, all of which are incorporated by reference herein in their entirety.

In a preferred embodiment of the invention, the DNA to be analyzed is detectably labeled, preferably by end-labeling. In a preferred embodiment of the invention, the DNA is labeled with a fluorescent group. Non-limiting examples of fluorescent groups suitable for use with the instant invention include 5-carboxyfluorescein (5-FAM), 6-carboxyfluorescein (6-FAM), 2′,7′-dimethoxy-4′,5′-dichloro-6-carboxyfluorescein (JOE), N,N,N′-N-tetramethyl-6-carboxy rhodamine (TAMRA), 6-carboxy-X-rhodamine (ROX), 4,7,2′,4′,5′,7′-hexachloro-6-carboxy-fluorescein (HEX-1), 4,7,2′,4′,5′, 7′-hexachloro-5-carboxy-fluorescein (HEX-2), 2′,4′,5′,7′-tetrachloro-5-carboxy-fluorescein (ZOE), 4,7,2′,7′-tetrachloro-6-carboxy-fluorescein (TET-1), 1′,2′,7′,8′-dibenzo-4,7-dichloro-5-carboxyfluorescein (NAN-2), and 1′,2′,7′, 8′-dibenzo-4,7-dichloro-6-carboxyfluorescein, fluorescein and fluorescein derivatives, Rhodamine, Cascade Blue, Alexa₃₅₀, Alexa₄₈₈, , phycoerythrin, allo-phycocyanin, phycocyanin, rhodamine, Texas Red, EDANS, BODIPY dyes such as BODIPY-FL and BODIPY-TR-X, tetramethylrhodamine, Cy3 and Cy5, 5,6-caroxyfluorescein, fluorescein mono-derivatized with a linking functionality at either the 5 or 6 carbon position, including fluorescein-5-isothiocyanate, fluorescein-6-isothiocyanate (the -5- and -6-forms being referred to collectively as FITC), fluorescein-5-succinimidylcarboxylate, fluorescein-6-succinimidylcarboxylate, fluorescein-5-iodoacetamide, fluorescein-6-iodoacetamide, fluorescein-5-maleimide, and fluorescein-6-maleimide;, 2′,7′-dimethoxy-4′,5′-dichlorofluorescein mono-derivatized with a linking functionality at the 5 or 6 carbon position, including 2′,7′-dimethoxy4′,5′-dichlorofluorescein -5-succinimidylcarboxylate and 2,′,7′-dimethoxy-4′,5′-dichlorofluoescein -6-succinimidylcarboxylate (the -5- and -6-forms being referred to collectively as DDFCS), tetramethylrhodamine mono-derivatized with a linking functionality at either the 5 or 6 carbon position, including tetramethylrhodamine-5-isothiocyanate, tetramethylrhodamine -6-isothiocyanate (the -5- and -6-forms being referred to collectively as TMRITC), tetramethylrhodamine-5-iodoacetamide, tetramethylrhodamine-6-iodoacetamide, tetramethylrhodamine-5-succinimidylcarboxylate, tetramethylrhodamine-6-succinimidylcarboxylate, tetramethylrhodamine-5-maleimide, and tetramethylrhodamine-6-maleimide, rhodamine X derivatives having a disubstituted phenyl attached to the molecule's oxygen heterocycle, one of the substituents being a linking functionality attached to the 4′ or 5′ carbon (IUPAC numbering) of the phenyl, and the other being a acidic anionic group attached to the 2′ carbon, including Texas Red (tradename of Molecular Probes, Inc.), rhodamine X-5-isothiocyanate, rhodamine X-6-isothiocyanate, rhodamine X-5-iodoacetamide, rhodamine X-6-iodoacetamide, rhodamine X-5-succinimidylcarboxylate, rhodamine X-6-succinimidylcarboxylate, rhodamine X-5-maleimide, and rhodamine X-6-maleimide.

Fluorescent labels can be attached to DNA using standard procedures, e.g. for a review see Haugland, “Covalent Fluorescent Probes,” in Excited States of Biopolymers, Steiner, Ed. (Plenum Press, New York, 1983), incorporated by reference herein in its entirety. In a preferred embodiment of the invention, a fluorescent group can be covalently attached to a desired primer by reaction with a 5′-amino-modified oligonucleotide in the presence of sodium bicarbonate and dimethylformamide, as described in U.S. patent application Ser. No. 09/169,440. Alternatively, the reactive amine can be attached by means of the linking agents disclosed in U.S. Pat. No. 4,757,141. Alternatively, covalently tagged primers can be obtained commercially (e.g., from Midland Certified Reagent, Co.). Fluorescent dyes are available form Molecular Probes, Inc. (Eugene, Oreg.), Operon Technologies, Inc., (Alameda, Calif.) and Amersham Pharmacia Biotech (Piscataway, N.J.), or can be synthesized using standard techniques. Fluorescent labeling is described in U.S. Pat. No. 4,855,225. Alternatively, the substrate can be end-labeled using T4 polynucleotide kinase and [γ-³²P]ATP, or with other reagents, such as biotin or digoxigenin depending on the particular detection and quantification system to be employed.

The DNA to be analyzed in the present invention can be obtained in purified form by any method known in the art. Any cell or virus can potentially serve as the nucleic acid source. The DNA may be obtained by standard procedures known in the art from cloned DNA, from amplified DNA, or directly from the desired cells (see, for example, Ausubel and Sambrook, cited supra). By way of example but not limitation, high molecular weight DNA can be isolated from eukaryotic cells by detergent lysis of cells followed by proteinase K digestion, phenol extraction, dialysis, density gradient centrifugation, and dialysis. Alternatively, cDNA reverse transcribed from mRNA, and optionally amplified (i.e., RT-PCR) can be analyzed using the present invention.

If it is desired to amplify any of the isolated DNA or a specific portion thereof, polymerase chain reaction (PCR) can be employed (U.S. Pat. Nos. 4,683,202, 4,683,195 and 4,889,818; Gyllenstein et al. (1988) Proc. Natl. Acad. Sci. USA 85:7652-7656; Ochman et al. (1988) Genetics 120:621-623; Loh et al. (1989) Science 243:217-220, which are incorporated herein by reference). Further guidance in determining an optimal amplification protocol can be found, for example, in Gelfand et al., PCR Protocols: A Guide to Methods and Applications, Academic Press (1990; ISBN: 0123721814) and Innis et al. PCR Applications: Protocols for Functional Genomics, Academic Press (1990; ISBN: 0123721857). Moreover, any of a variety of nucleic acid amplification and other molecular biology techniques known to the skilled artisan can be used to generate the desired DNA segments for analysis pursuant to the present invention.

Oligonucleotides can be prepared by any suitable method, including, for example, cloning and restriction of appropriate sequences and direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; and the solid support method of U.S. Pat. No. 4,458,066, each incorporated herein by reference. A review of synthesis methods is provided in Goodchild (1990) Bioconjugate Chemistry 1(3):165-187, incorporated herein by reference.

In one aspect of the invention, a chromatographic DNA sequencing ladder is generated using one or more base-discriminating cleavage reactions, e.g. one of the Maxam-Gilbert chemical sequencing reactions, or a modified version of one of these reactions. In a preferred embodiment of the invention, only a single base-discriminating cleavage reaction is used, e.g., the G+A sequencing reaction. In a particularly preferred embodiment of the invention, the express protocol for generating a G+A sequencing ladder described by Belikov and Wieslander is employed (Belikov & Wieslander (1995) Nucleic Acids Res. 23:310-11). This procedure is easier and requires less time that the original Maxam-Gilbert sequencing reaction, and is based on partial acidic hydrolyses of DNA in the presence of diphenylamine and proceeds via depurination/5′,3′-elimination.

In a typical G+A sequencing reaction performed according to Belikov and Wieslander, 10 μL of diphenylamine (Aldrich) in formic acid (Aldrich) is added to 75 pmol of an oligodeoxynucleotide that contains a 5′-fluorescent group, MilliQ water is added to bring the sample up to 20 μL, and the reaction is incubated at room temperature for 10-20 minutes. If desired, carrier DNA can be added. 100 μL of 0.3 M sodium acetate (pH 5.5) is added to stop the reaction, after which the mixture is extracted three times with water-saturated ether. The sample is then placed in a vacuum dryer to remove traces of ether and precipitated by the addition of 3 volumes of ethanol and placed at −20° C. for 30 minutes. The DNA is then centrifuged for 15 minutes at 15,000×g and resuspended in 20 μL MilliQ water. 2-5 μL is used to generate a chromatographic DNA sequencing ladder.

An important element of the instant invention that makes it superior to previously available sequencing ladder is the use of high performance liquid chromatography (HPLC) rather than electrophoresis to separate and detect the DNA fragments. The use of HPLC instead of electrophoresis results in a number of advantages, including shorter analysis times, more reproducible data, convenience, ease of use, improved capability for high-throughput and automation, enhanced ability to resolve and detect very small DNA fragments.

In preferred embodiment of the invention ion pairing reverse phase HPLC (IP-RP-HPLC) is used to analyze the DNA cleavage products. IP-RP-HPLC is a form of chromatography particularly suited to the analysis of both single and double stranded polynucleotides, and is characterized by the use of a reverse phase (i.e., hydrophobic) stationary phase and a mobile phase that includes an alkylated cation (e.g., triethylammonium) that is believed to form a bridging interaction between the negatively charged DNA and non-polar stationary phase. The alkylated cation-mediated interaction of DNA and stationary phase can be modulated by the polarity of the mobile phase, conveniently adjusted by means of a solvent that is less polar than water, e.g., acetonitrile. Performance is enhanced by the use of a non-porous separation medium, as described in U.S. patent application No. 5,585,236. It has been shown that under non-denaturing conditions the retention time of a double-stranded DNA fragment is dictated by the size of the fragment; the base composition or sequence of the fragment does not appreciably affect the separation. The most preferred method of analysis by means of Matched Ion Polynucleotide Chromatography (MIPC), a superior form of IP-RP-HPLC described in U.S. Pat. Nos. 5,585,236, 6,066,258 and 6,056,877 and PCT Publication Nos. WO98/48913, WO98/48914, WO/9856797, WO98/56798, incorporated herein by reference in their entirety. MIPC is characterized by the use of solvents and chromatographic surfaces that are substantially free of multivalent cation contamination that can interfere with polynucleotide separation. In the practice of the invention, a preferred system for performing MIPC separations is that provided by Transgenomic, Inc. (San Jose, Calif.) under the trademark WAVE®. The highly reproducible nature of IP-RP-HPLC, and MIPC in particular, lends itself to the use of a DNA sequencing ladder to phase a reaction, since the elution times of corresponding fragments should remain relatively constant for the parallel runs.

Separation by RP-IP-HPLC, including MIPC, occurs at the non-polar surface of a separation medium. In one embodiment, the non-polar surfaces comprise the surfaces of polymeric beads. In an alternative embodiment, the surfaces comprise the surfaces of interstitial spaces in a molded polymeric monolith, described in more detail infra. For purposes of simplifying the description of the invention and not by way of limitation, the separation of polynucleotides using nonporous beads, and the preparation of such beads, will be primarily described herein, it being understood that other separation surfaces, such as the interstitial surfaces of polymeric monoliths, are intended to be included within the scope of this invention.

In general, in order to be suitable for use in IP-RP-HPLC a separation medium should have a surface that is either intrinsically non-polar or bonded with a material that forms a surface having sufficient non-polarity to interact with a counterion agent.

In one aspect of the invention, IP-RP-HPLC detection is accomplished using a column filled with nonporous polymeric beads having an average diameter of about 0.5-100 microns; preferably, 1-10 microns; more preferably, 1-5 microns. Beads having an average diameter of 1.0-3.0 microns are most preferred.

In a preferred embodiment of the invention, the chromatographic separation medium comprises nonporous beads, i.e., beads having a pore size that essentially excludes the polynucleotides being separated from entering the bead, although porous beads can also be used. As used herein, the term “nonporous” is defined to denote a bead that has surface pores having a diameter that is sufficiently small so as to effectively exclude the smallest DNA fragment in the separation in the solvent medium used therein. Included in this definition are polymer beads having these specified maximum size restrictions in their natural state or which have been treated to reduce their pore size to meet the maximum effective pore size required.

The surface conformations of nonporous beads of the present invention can include depressions and shallow pit-like structures that do not interfere with the separation process. A pretreatment of a porous bead to render it nonporous can be effected with any material which will fill the pores in the bead structure and which does not significantly interfere with the MIPC process.

Pores are open structures through which mobile phase and other materials can enter the bead structure. Pores are often interconnected so that fluid entering one pore can exit from another pore. Without intending to be bound by any particular theory, it is believed that pores having dimensions that allow movement of the polynucleotide into the interconnected pore structure and into the bead impair the resolution of separations or result in separations that have very long retention times.

Non-porous polymeric beads useful in the practice of the present invention can be prepared by a two-step process in which small seed beads are initially produced by emulsion polymerization of suitable polymerizable monomers. The emulsion polymerization procedure is a modification of the procedure of Goodwin, et al. (Colloid & Polymer Sci., 252:464471 (1974)). Monomers which can be used in the emulsion polymerization process to produce the seed beads include styrene, alkyl substituted styrenes, alpha-methyl styrene, and alkyl substituted alpha-methyl styrene. The seed beads are then enlarged and, optionally, modified by substitution with various groups to produce the nonporous polymeric beads of the present invention.

The seed beads produced by emulsion polymerization can be enlarged by any known process for increasing the size of the polymer beads. For example, polymer beads can be enlarged by the activated swelling process disclosed in U.S. Pat. No. 4,563,510. The enlarged or swollen polymer beads are further swollen with a crosslinking polymerizable monomer and a polymerization initiator. Polymerization increases the crosslinking density of the enlarged polymeric bead and reduces the surface porosity of the bead. Suitable crosslinking monomers contain at least two carbon-carbon double bonds capable of polymerization in the presence of an initiator. Preferred crosslinking monomers are divinyl monomers, preferably alkyl and aryl (phenyl, naphthyl, etc.) divinyl monomers and include divinyl benzene, butadiene, etc. Activated swelling of the polymeric seed beads is useful to produce polymer beads having an average diameter ranging from 1 up to about 100 microns.

Alternatively, the polymer seed beads can be enlarged simply by heating the seed latex resulting from emulsion polymerization. This alternative eliminates the need for activated swelling of the seed beads with an activating solvent. Instead, the seed latex is mixed with the crosslinking monomer and polymerization initiator described above, together with or without a water-miscible solvent for the crosslinking monomer. Suitable solvents include acetone, tetrahydrofuran (THF), methanol, and dioxane. The resulting mixture is heated for about 1-12 hours, preferably about 4-8 hours, at a temperature below the initiation temperature of the polymerization initiator, generally, about 10° C.-80° C., preferably 30° C.-60° C. Optionally, the temperature of the mixture can be increased by 10-20% and the mixture heated for an additional 1 to 4 hours. The ratio of monomer to polymerization initiator is at least 100:1, preferably in the range of about 100:1 to about 500:1, more preferably about 200:1 in order to ensure a degree of polymerization of at least 200. Beads having this degree of polymerization are sufficiently pressure-stable to be used in HPLC applications. This thermal swelling process allows one to increase the size of the bead by about 110-160% to obtain polymer beads having an average diameter up to about 5 microns, preferably about 2-3 microns. The thermal swelling procedure can, therefore, be used to produce smaller particle sizes previously accessible only by the activated swelling procedure.

Following thermal enlargement, excess crosslinking monomer is removed and the particles are polymerized by exposure to ultraviolet light or heat. Polymerization can be conducted, for example, by heating of the enlarged particles to the activation temperature of the polymerization initiator and continuing polymerization until the desired degree of polymerization has been achieved. Continued heating and polymerization allows one to obtain beads having a degree of polymerization greater than 500.

For use in the present invention, packing material disclosed by U.S. Pat. No. 4,563,510 can be modified through substitution of the polymeric beads with alkyl groups or can be used in its unmodified state. For example, the polymer beads can be alkylated with 1 or 2 carbon atoms by contacting the beads with an alkylating agent, such as methyl iodide or ethyl iodide. Alkylation can be achieved by mixing the polymer beads with the alkyl halide in the presence of a Friedel-Crafts catalyst to effect electrophilic aromatic substitution on the aromatic rings at the surface of the polymer blend. Suitable Friedel-Crafts catalysts are well-known in the art and include Lewis acids such as aluminum chloride, boron trifluoride, tin tetrachloride, etc. The beads can be hydrocarbon substituted by substituting the corresponding hydrocarbon halide for methyl iodide in the above procedure, for example.

The term alkyl as used herein in reference to the beads useful in the practice of the present invention is defined to include alkyl and alkyl substituted aryl groups, having from 1 to 1,000,000 carbons, the alkyl groups including straight chained, branch chained, cyclic, saturated, unsaturated nonionic functional groups of various types including aldehyde, ketone, ester, ether, alkyl groups, and the like, and the aryl groups including as monocyclic, bicyclic, and tricyclic aromatic hydrocarbon groups including phenyl, naphthyl, and the like. Methods for alkyl substitution are conventional and well-known in the art and are not an aspect of this invention. The substitution can also contain hydroxy, cyano, nitro groups, or the like which are considered to be non-polar, reverse phase functional groups.

Non-limiting examples of base polymers suitable for use in producing such polymer beads include mono- and di-vinyl substituted aromatics such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(styrene-divinylbenzene) and poly(ethylvinylbenzene-divinylbenzene). Methods for making beads from these polymers are conventional and well known in the art (for example, see U.S. Pat. No. 4,906,378). The physical properties of the surface and near-surface areas of the beads are the primary determinant of chromatographic efficiency. The polymer, whether derivatized or not, should provide a nonporous, non-reactive, and non-polar surface for the MIPC separation. In a particularly preferred embodiment of the invention, the separation medium consists of octadecyl modified, nonporous alkylated poly(styrene-divinylbenzene) beads. Separation columns employing these particularly preferred beads, referred to as DNASep® columns, are commercially available from Transgenomic, Inc.

A separation bead used in the invention can comprise a nonporous particle which has non-polar molecules or a non-polar polymer attached to or coated on its surface. In general, such beads comprise nonporous particles which have been coated with a polymer or which have substantially all surface substrate groups reacted with a non-polar hydrocarbon or substituted hydrocarbon group, and any remaining surface substrate groups endcapped with a tri(lower alkyl)chlorosilane or tetra(lower alkyl)dichlorodisilazane as described in U.S Pat. No. 6,056,877.

The nonporous particle is preferably an inorganic particle, but can be a nonporous organic particle. The nonporous particle can be, for example, silica, silica carbide, silica nitrite, titanium oxide, aluminum oxide, zirconium oxide, carbon, insoluble polysaccharides such as cellulose, or diatomaceous earth, or any of these materials which have been modified to be nonporous. Examples of carbon particles include diamond and graphite which have been treated to remove any interfering contaminants. The preferred particles are essentially non-deformable and can withstand high pressures. The nonporous particle is prepared by known procedures. The preferred particle size is about 0.5-100 microns; preferably, 1-10 microns; more preferably, 1-5 microns. Beads having an average diameter of 1.0-3.0 microns are most preferred.

Because the chemistry of preparing conventional silica-based reverse phase HPLC materials is well-known, most of the description of non-porous beads suitable for use in the instant invention is presented in reference to silica. It is to be understood, however, that other nonporous particles, such as those listed above, can be modified in the same manner and substituted for silica. For a description of the general chemistry of silica, see Poole, Colin F. and Salwa K. Poole, Chromatography Today, Elsevier:New York (1991), pp. 313-342 and Snyder, R. L. and J. J. Kirkland, Introduction to Modern Liquid Chromatography, 2^{nd} ed., John Wiley & Sons, Inc.:New York (1979), pp.272-278, the disclosures of which are hereby incorporated herein by reference in their entireties.

The nonporous beads of the invention are characterized by having minimum exposed silanol groups after reaction with the coating or silating reagents. Minimum silanol groups are needed to reduce the interaction of the DNA with the substrate and also to improve the stability of the material in a high pH and aqueous environment. Silanol groups can be harmful because they can repel the negative charge of the DNA molecule, preventing or limiting the interaction of the DNA with the stationary phase of the column. Another possible mechanism of interaction is that the silanol can act as ion exchange sites, taking up metals such as iron (III) or chromium (III). Iron (III) or other metals which are trapped on the column can distort the DNA peaks or even prevent DNA from being eluted from the column.

Silanol groups can be hydrolyzed by the aqueous-based mobile phase. Hydrolysis will increase the polarity and reactivity of the stationary phase by exposing more silanol sites, or by exposing metals that can be present in the silica core. Hydrolysis will be more prevalent with increased underivatized silanol groups. The effect of silanol groups on the DNA separation depends on which mechanism of interference is most prevalent. For example, iron (III) can become attached to the exposed silanol sites, depending on whether the iron (III) is present in the eluent, instrument or sample.

The effect of metals can only occur if metals are already present within the system or reagents. Metals present within the system or reagents can get trapped by ion exchange sites on the silica. However, if no metals are present within the system or reagents, then the silanol groups themselves can cause interference with DNA separations. Hydrolysis of the exposed silanol sites by the aqueous environment can expose metals that might be present in the silica core.

Fully hydrolyzed silica contains a concentration of about 8 μmoles of silanol groups per square meter of surface. At best, because of steric considerations, a maximum of about 4.5 μmoles of silanol groups per square meter can be reacted, the remainder of the silanol being sterically shielded by the reacted groups. Minimum silanol groups is defined as reaching the theoretical limit of or having sufficient shield to prevent silanol groups from interfering with the separation.

Numerous methods exist for forming nonporous silica core particles. For example, sodium silicate solution poured into methanol will produce a suspension of finely divided spherical particles of sodium silicate. These particles are neutralized by reaction with acid. In this way, globular particles of silica gel are obtained having a diameter of about 1-2 microns. Silica can be precipitated from organic liquids or from a vapor. At high temperature (about 2000° C.), silica is vaporized, and the vapors can be condensed to form finely divided silica either by a reduction in temperature or by using an oxidizing gas. The synthesis and properties of silica are described by R. K. IIer in The Chemistry of Silica, Solubility, Polymerization, Colloid and Surface Properties, and Biochemistry, John Wiley & Sons:New York (1979).

W. Stöber et al. described controlled growth of monodisperse silica spheres in the micron size range in J. Colloid and Interface Sci., 26:62-69 (1968). Stöber et al. describe a system of chemical reactions which permit the controlled growth of spherical silica particles of uniform size by means of hydrolysis of alkyl silicates and subsequent condensation of silicic acid in alcoholic solutions. Ammonia is used as a morphological catalyst. Particle sizes obtained in suspension range from less than 0.05 μm to 2 μm in diameter.

To prepare a nonporous bead, the nonporous particle can be coated with a polymer or reacted and endcapped so that substantially all surface substrate groups of the nonporous particle are blocked with a non-polar hydrocarbon or substituted hydrocarbon group. This can be accomplished by any of several methods described in U.S. Pat. No. 6,056,877. Care should be taken during the preparation of the beads to ensure that the surface of the beads has minimum silanol or metal oxide exposure and that the surface remains nonporous. Nonporous silica core beads can be obtained from Micra Scientific (Northbrook, Ill.) and from Chemie Uetikkon (Lausanne, Switzerland).

In another embodiment of the present invention, the IP-RP-HPLC separation medium can be in the form of a polymeric monolith, e.g., a rod-like monolithic column. A monolith is a polymer separation media, formed inside a column, having a unitary structure with through pores or interstitial spaces that allow eluting solvent and analyte to pass through and which provide the non-polar separation surface, as described in U.S. Pat. No. 6,066,258 and U.S. patent application Ser. No. 09/562,069. The interstitial separation surfaces can be porous, but are preferably nonporous. The separation principles involved parallel those encountered with bead-packed columns. As with beads, pores traversing the monolith must be compatible with and permeable to DNA. In a preferred embodiment, the rod is substantially free of contamination capable of reacting with DNA and interfering with its separation, e.g., multivalent cations.

A molded polymeric monolith rod that can be used in practicing the present invention can be prepared, for example, by bulk free radical polymerization within the confines of a chromatographic column. The base polymer of the rod can be produced from a variety of polymerizable monomers. For example, the monolithic rod can be made from polymers, including mono- and di-vinyl substituted aromatic compounds such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(glycidyl methacrylate-co-ethylene dimethacrylate), poly(styrene-divinylbenzene) and poly(ethylvinylbenzene-divinylbenzene. The rod can be unsubsituted or substituted with a substituent such as a hydrocarbon alkyl or an aryl group. The alkyl group optionally has 1 to 1,000,000 carbons inclusive in a straight or branched chain, and includes straight chained, branch chained, cyclic, saturated, unsaturated nonionic functional groups of various types including aldehyde, ketone, ester, ether, alkyl groups, and the like, and the aryl groups includes as monocyclic, bicyclic, and tricyclic aromatic hydrocarbon groups including phenyl, naphthyl, and the like. In a preferred embodiment, the alkyl group has 1-24 carbons. In a more preferred embodiment, the alkyl group has 1-8 carbons. The substitution can also contain hydroxy, cyano, nitro groups, or the like which are considered to be non-polar, reverse phase functional groups. Methods for hydrocarbon substitution are conventional and well-known in the art and are not an aspect of this invention. The preparation of polymeric monoliths is by conventional methods well known in the art as described in the following references: Wang et al.(1994) J. Chromatog. A 699:230; Petro et al. (1996) Anal. Chem. 68:315 and U.S. Pat. Nos. 5,334,310; 5,453,185 and 5,522,994. Monolith or rod columns are commercially available form Merck & Co (Darmstadt, Germany).

The separation medium can take the form of a continuous monolithic silica gel. A molded monolith can be prepared by polymerization within the confines of a chromatographic column (e.g., to form a rod) or other containment system. A monolith is preferably obtained by the hydrolysis and polycondensation of alkoxysilanes. A preferred monolith is derivatized in order to produce non-polar interstitial surfaces. Chemical modification of silica monoliths with ocatdecyl, methyl or other ligands can be carried out. An example of a preferred derivatized monolith is one which is polyfunctionally derivatized with octadecylsilyl groups. The preparation of derivatized silica monoliths can be accomplished using conventional methods well known in the art as described in the following references which are hereby incorporated in their entirety herein: U.S Pat. No. 6,056,877, Nakanishi, et al., J. Sol-Gel Sci. Technol. 8:547 (1997); Nakanishi, et al., Bull, Chem. Soc. Jpn. 67:1327 (1994); Cabrera, et al., Trends Analytical Chem. 17:50 (1998); Jinno, et al., Chromatographia 27:288 (1989).

MIPC is characterized by the use of a separation medium having low amounts of metal contaminants or other contaminants that can bind DNA. Preferred beads and monoliths have been produced under conditions where precautions have been taken to substantially eliminate any multivalent cation contaminants (e.g. Fe(III), Cr(III), or colloidal metal contaminants), including a decontamination treatment, e.g., an acid wash treatment. Only very pure, non-metal containing materials should be used in the production of the beads in order to minimize the metal content of the resulting beads.

In addition to the separation medium being substantially metal-free, to achieve optimum peak separation the separation column and all process solutions held within the column or flowing through the column are preferably substantially free of multivalent cation contaminants (e.g. Fe(III), Cr(III), and colloidal metal contaminants). As described in U.S. Pat. No. 5,772,889, 5,997,742 and 6,017,457, this can be achieved by supplying and feeding solutions that enter the separation column with components that have process solution-contacting surfaces made of material which does not release multivalent cations into the process solutions held within or flowing through the column, in order to protect the column from multivalent cation contamination. The process solution-contacting surfaces of the system components are preferably material selected from the group consisting of titanium, coated stainless steel, passivated stainless steel, and organic polymer. Metals found in stainless steel, for example, do not harm the separation, unless they are in an oxidized or colloidal partially oxidized state. For example, 316 stainless steel frits are acceptable in column hardware, but surface oxidized stainless steel frits harm the DNA separation.

For additional protection, multivalent cations in mobile phase solutions and sample solutions entering the column can be removed by contacting these solutions with multivalent cation capture resin before the solutions enter the column to protect the separation medium from multivalent cation contamination. The multivalent capture resin is preferably cation exchange resin and/or chelating resin.

Trace levels of multivalent cations anywhere in the solvent flow path can cause a significant deterioration in the resolution of the separation after multiple uses of an IP-RP-HPLC column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the MIPC column. These, and similar measures, taken to prevent system contamination with multivalent cations have resulted in extended column life and reduced analysis downtime.

There are two places where multivalent-cation-binding agents, e.g., chelators, are used in MIPC separations. In one embodiment, these binding agents can be incorporated into a solid through which the mobile phase passes. Contaminants are trapped before they reach places within the system that can harm the separation. In these cases, the functional group is attached to a solid matrix or resin (e.g., a flow-through cartridge, usually an organic polymer, but sometimes silica or other material). The capacity of the matrix is preferably about 2 mequiv./g. An example of a suitable chelating resin is available under the trademark CHELEX 100 (Dow Chemical Co.) containing an iminodiacetate functional group.

In another embodiment, the multivalent cation-binding agent can be added to the mobile phase. The binding functional group is incorporated into an organic chemical structure. The preferred multivalent cation-binding agent fulfills three requirements. First, it is soluble in the mobile phase. Second, the complex with the metal is soluble in the mobile phase. Multivalent cation-binding agents such as EDTA fulfill this requirement because both the chelator and the multivalent cation-binding agent-metal complex contain charges, which makes them both water-soluble. Also, neither precipitate when acetonitrile, for example, is added. The solubility in aqueous mobile phase can be enhanced by attaching covalently bound ionic functionality, such as, sulfate, carboxylate, or hydroxy. A preferred multivalent cation-binding agent can be easily removed from the column by washing with water, organic solvent or mobile phase. Third, the binding agent must not interfere with the chromatographic process.

The multivalent cation-binding agent can be a coordination compound. Examples of preferred coordination compounds include water soluble chelating agents and crown ethers. Non-limiting examples of multivalent cation-binding agents which can be used in the present invention include acetylacetone, alizarin, aluminon, chloranilic acid, kojic acid, morin, rhodizonic acid, thionalide, thiourea, α-furildioxime, nioxime, salicylaldoxime, dimethylglyoxime, α-furildioxime, cupferron, α-nitroso-β-naphthol, nitroso-R-salt, diphenylthiocarbazone, diphenylcarbazone, eriochrome black T, PAN, SPADNS, glyoxal-bis(2-hydroxyanil), murexide, α-benzoinoxime, mandelic acid, anthranilic acid, ethylenediamine, glycine, triaminotriethylamine, thionalide, triethylenetetrarmine, EDTA, metalphthalein, arsonic acids, α,α′-bipyridine, 4-hydroxybenzothiazole, 8-hydroxyquinaldine, 8-hydroxyquinoline, 1,10-phenanthroline, picolinic acid, quinaldic acid, α,α′,α″-terpyridyl, 9-methyl-2,3,7-trihydroxy-6-fluorone, pyrocatechol, salicylic acid, tiron, 4-chloro-1,2-dimercaptobenzene, dithiol, mercaptobenzothiazole, rubeanic acid, oxalic acid, sodium diethyldithiocarbarbamate, and zinc dibenzyldithiocarbamate. These and other examples are described by Perrin in Organic Complexing Reagents: Structure, Behavior, and Application to Inorganic Analysis, Robert E. Krieger Publishing Co. (1964). In the present invention, a preferred multivalent cation-binding agent is EDTA.

To achieve high-resolution chromatographic separations of polynucleotides, it is generally necessary to tightly pack the chromatographic column with the solid phase polymer beads. Any known method of packing the column with a column packing material can be used in the present invention to obtain adequate high-resolution separations. Typically, a slurry of the polymer beads is prepared using a solvent having a density equal to or less than the density of the polymer beads. The column is then filled with the polymer bead slurry and vibrated or agitated to improve the packing density of the polymer beads in the column. Mechanical vibration or sonication is typically used to improve packing density.

For example, to pack a 50×4.6 mm I.D. column, 2.0 grams of beads can be suspended in 10 mL of methanol with the aid of sonication. The suspension is then packed into the column using 50 mL of methanol at 8,000 psi of pressure. This improves the density of the packed bed.

There are several types of counterions suitable for use with IP-RP-HPLC. These include a mono-, di-, or trialkylamine that can be protonated to form a positive counter charge or a quaternary alkyl substituted amine that already contains a positive counter charge. The alkyl substitutions may be uniform (for example, triethylammonium acetate or tetrapropylammonium acetate) or mixed (for example, propyidiethylammonium acetate). The size of the alkyl group may be small (methyl) or large (up to 30, carbons) especially if only one of the substituted alkyl groups is large and the others are small. For example octyidimethylammonium acetate is a suitable counterion agent. Preferred counterion agents are those containing alkyl groups from the ethyl, propyl or butyl size range.

Without intending to be bound by any particular theory, it is believed the alkyl group functions by imparting a nonpolar character to the DNA through an ion pairing process so that the DNA can interact with the nonpolar surface of the separation media. The requirements for the degree of nonpolarity of the counterion-DNA pair depends on the polarity of the separation media, the solvent conditions required for separation, the particular size and type of fragment being separated. For example, if the polarity of the separation media is increased, then the polarity of the counterion agent may have to be adjusted to match the polarity of the surface and increase interaction of the counterion-DNA pair. In general, as the size and hydrophobicity of the alkyl group is increased, the separation is less influenced by DNA sequence and base composition, but rather is based predominately on DNA sequence length.

In some cases, it may be desired to increase the range of concentration of organic solvent used to perform the separation. For example, increasing the alkyl chain length on the counterion agent will increase the nonpolarity of the counterion-DNA pair resulting in the need to either increase the concentration of the mobile phase organic solvent, or increase the strength of the organic solvent type, e.g., acetonitrile is about two times more effective than methanol for eluting DNA. There is a positive correlation between concentration of the organic solvent required to elute a fragment from the column and the length of the fragment. However, at high organic solvent concentrations, the polynucleotide can precipitate. To avoid precipitation, a more non-polar organic solvent and/or a smaller counterion alkyl group can be used. The alkyl group on the counterion reagent can also be substituted with halides, nitro groups, or the like to modulate polarity.

The mobile phase preferably contains a counterion agent. Typical counterion agents include trialkylammonium salts of organic or inorganic acids, such as lower alkyl primary, secondary, and lower tertiary amines, lower trialkyammonium salts and lower quaternary alkyalmmonium salts. Lower alkyl refers to an alkyl radical of one to six carbon atoms, as exemplified by methyl, ethyl, n-butyl, i-butyl, t-butyl, isoamyl, n-pentyl, and isopentyl. Examples of counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, and tetrabutylammonium acetate. Although the anion in the above examples is acetate, other anions may also be used, including carbonate, phosphate, sulfate, nitrate, propionate, formate, chloride, and bromide, or any combination of cation and anion. These and other agents are described by Gjerde, et al. in Ion Chromatography, 2nd Ed., Dr. Alfred Hüthig Verlag Heidelberg (1987). In a particularly preferred embodiment of the invention the counterion is tetrabutylammonium bromide (TBAB) is preferred, although other quaternary ammonium reagents such as tetrapropyl or tetrabutyl ammonium salts can be used. Alternatively, a trialkylammonium salt, e.g., triethylammonium acetate (TEAA) can be used.

The pH of the mobile phase is preferably within the range of about pH 5 to about pH 9, and optimally within the range of about pH 6 to about pH 7.5.

To achieve optimum peak resolution during the separation of DNA by IP-RP-HPLC, the method is preferably performed at a temperature within the range of 20° C. to 90° C.; more preferably, 30° C. to 80° C.; most preferably, 50° C. to 75° C. The flow rate is selected to yield a back pressure not exceeding 5000 psi. In general, separation of single-stranded fragments should be performed at higher temperatures. In a preferred embodiment of the invention, the separation is achieved at a temperature at which the amplified extension product is denatured. The temperature required to achieve denaturation will vary, depending upon the nature of the column, the mobile phase and counterion agent used, and the melting properties of the DNA being separated. In a particularly preferred embodiment of the invention, where the separation medium is octadecyl modified, nonporous alkylated poly(styrene-divinylbenzene) beads, the aqueous mobile phase contains acetonitrile and TBAB is used as a counterion, the column temperature is preferably greater than 50° C., more preferably between about 50° C. and 80° C., and most preferably about 70° C.

The temperature at which the separation is performed affects the choice of organic solvents used in the separation, and vice versa. The solvent affects the temperature at which a double stranded DNA will melt to form two single strands or a partially melted complex of single and double stranded DNA, i.e., some solvents will stabilize a DNA duplex better than others. Furthermore, the polarity of a solvent affects the distribution of the DNA between the mobile phase and the stationary phase.

An organic solvent that is water soluble is preferably used, e.g., an alcohol, nitrile, dimethylformamide (DMF), tetrahydrofuran (THF), ester, or ether. Water soluble solvents are defined as those that exist as a single phase with aqueous systems under all conditions of operation of the present invention. For example, acetonitrile and 1-propanol have polarity and solubility properties that are particularly suited for use in the present invention. However, methanol can be a good alternative that reduces cost and toxicity concerns. Solvents that are particularly preferred for use in the method of this invention include methanol, ethanol, 2-propanol, 1-propanol, tetrahydrofuran (THF), and acetonitrile, with acetonitrile being most preferred overall.

In performing IP-RP-HPLC and MIPC, even trace levels of multivalent cations anywhere in the solvent flow path can cause a significant deterioration in the resolution of the separation after multiple uses of a column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the column. These, and similar measures, taken to prevent system contamination with multivalent cations have resulted in extended column life and reduced analysis downtime.

In some instances, in order to optimize column life and maintain effective separation performance, it will be desirable to periodically run an aqueous solution of multivalent cation-binding agent through the column, e.g., after about 500 uses or when the performance starts to degrade. Examples of suitable cation-binding agents are as described hereinabove.

The concentration of a solution of the cation-binding agent can be between 0.01M and 1M. In a preferred embodiment, the column washing solution contains EDTA at a concentration of about 0.03 to 0.1M.

In another embodiment, the solution contains an organic solvent selected from the group consisting of acetonitrile, ethanol, methanol, 2-propanol, and ethyl acetate. A preferred solution contains at least 2% organic solvent to prevent microbial growth. In a most preferred embodiment a solution containing 25% acetonitrile is used to wash a column. The multivalent cation-binding solution can contain a counterion agent as described hereinabove.

In one embodiment of a column washing procedure, the separation column is washed with the multivalent cation-binding solution at an elevated temperature in the range of 50° C. to 80° C. In a preferred embodiment the column is washed with a solution containing EDTA, TEAA, and acetonitrile, in the 70° C. to 80° C. temperature range. In a specific embodiment, the solution contains 0.032 M EDTA, 0.1M TEAA, and 25% acetonitrile.

Column washing can range from 30 seconds to one hour. In a preferred procedure, the column is washed with multivalent cation-binding agent for 30 to 60 minutes at a flow rate preferably in the range of about 0.05 to 1.0 mL/min.

Other treatments for washing a column can also be used alone or in combination with those indicated hereinabove. These include: use of high pH washing solutions (e.g., pH 10-12), use of denaturants such as urea or formamide, and reverse flushing the column with washing solution.

MIPC separation efficiency can be preserved by storing the column separation media in the presence of a solution of multivalent cation-binding agent. The solution of binding agent may also contain a counterion agent. Any of the multivalent cation-binding agents, counterion agents, and solvents described hereinabove are suitable for the purpose of storing a MIPC column. In a preferred embodiment, a column packed with MIPC separation media is stored in an organic solvent containing a multivalent cation-binding agent and a counterion agent. An example of this preferred embodiment is 0.032 M EDTA and 0.1 M TEM in 25% aqueous acetonitrile. In preparation for storage, a solution of multivalent cation-binding agent, as described above, is passed through the column for about 30 minutes. The column is then disconnected from the HPLC apparatus and the column ends are capped with commercially available threaded end caps made of material which does not release multivalent cations. Such end caps can be made of coated stainless steel, titanium, organic polymer or any combination thereof.

High pressure pumps are used for pumping mobile phase in the systems described in U.S. Pat. No. 5,585,236 to Bonn and in U.S. Pat. No. 5,772,889 to Gjerde. It will be appreciated that other methods are known for driving mobile phase through separation media and can be used in carrying out the analysis described in the present invention. A non-limiting example of such an alternative method includes “capillary electrochromatography” (CEC) in which an electric field is applied across capillary columns packed with microparticles and the resulting electroosmotic flow acts as a pump for chromatography. Electroosmosis is the flow of liquid, in contact with a solid surface, under the influence of a tangentially applied electric field. The technique combines the advantages of the high efficiency obtained with capillary electrophoretic separations, such as capillary zone electrophoresis, and the general applicability of HPLC. CEC has the capability to drive the mobile phase through columns packed with chromatographic particles, especially small particles, when using electroosmotic flow. High efficiencies can be obtained as a result of the plug-like flow profile. In the use of CEC in the present invention, solvent gradients are used and rapid separations can be obtained using high electric fields. The following references describing CEC are each incorporated in their entirety herein: Dadoo, et al, LC-GC 15:630 (1997); Jorgenson, et al., J. Chromatog. 218:209 (1981); Pretorius, et al., J. Chromatog. 99:23 (1974); and the following U.S. Pat. Nos. to Dadoo 5,378,334 (1995), 5,342,492 (1994), and 5,310,463 (1994). In the operation of this aspect of the present invention, the capillaries are packed, either electrokinetically or using a pump, with the separation beads described in the present specification. In another embodiment, a polymeric rod is prepared by bulk free radical polymerization within the confines of a capillary column. Capillaries are preferably formed from fused silica tubing or etched into a block. The packed capillary (e.g., a 150-μm i.d. with a 20-cm packed length and a window located immediately before the outlet frit) is fitted with frits at the inlet and outlet ends. An electric field, e.g., 2800V/cm, is applied. Detection can be by uv absorbance or by fluorescence. A gradient of organic solvent, e.g., acetonitrile, is applied in a mobile phase containing counterion agent (e.g. 0.1 M TEAA). to elute the polynucleotides. The column temperature is maintained by conventional temperature control means. In the preferred embodiment, all of the precautions for minimizing trace metal contaminants as described hereinabove are employed in using CEC.

Other features of the invention will become apparent in the course of the following descriptions of exemplary embodiments, which are given for illustration of the invention and are not intended to be limiting thereof.

Procedures described in the past tense in the examples below have been carried out in the laboratory. Procedures described in the present tense have not yet been carried out in the laboratory, and are constructively reduced to practice with the filing of this application.

### EXAMPLE 1

### Phasing a DNA Footprinting Experiment

The oligonucleotides used in this and subsequent examples were synthesized on an Applied Biosystems 394 DNA synthesiser using cyanoethyl phosphoramidite chemistry. Following deprotection, the oligonucleotides were purified using denaturing PAGE, evaporated to dryness and desalted using a Pharmacia NAP 10 column according to the manufacturer's instructions. 5 pmol of labeled synthetic Holliday junction HJ50 was prepared by annealing and purifying the four 50-mer oligonucleotides HJ1, HJ2, HJ3 and HJ4

| | |
|---|---|
| (HJ1 | (SEQ ID NO:1) |

5′GTCGGATCCTCTAGACAGCTCCATGTTCACTGGCACTGGTAGAATTCG
GC,

| | |
|---|---|
| HJ2 | (SEQ ID NO:2) |

5′-ACGTCATAGACGATTACATTGCTACATGGAGCTGTCTAGAGGATCC
GA;

| | |
|---|---|
| HJ3 | (SEQ ID NO:3) |

5′-(6-FAM)-TGCCGAATTCTACCAGTGCCAGTGCCAGTGATGGACATC
TTTGCCCACGTTGACCC and

| | |
|---|---|
| HJ4 | (SEQ ID NO:4) |

5′-(TET)-GGGTCAACGTGGGCAAGATGTCCTAGCAATGTAATCGTCTA
TGACGTT,)

essentially as described in Parsons et al. (1990) J Biol Chem 265:9285-9.

HJ50 was added to a solution of 100 mM Ascorbate (Aldrich), followed by 5 μl of 1.2% H₂O₂ (Aldrich), 10 μl of 20 mM Fe²⁺/40 mM EDTA (Aldrich) solution was added and rapidly mixed and incubated at room temp for 4 minutes. The reaction was then stopped by the addition of 10 μl of 0.1M thiourea (Sigma) and 0.1M EDTA solutions.

20 μl of this solution was then analyzed using IP-RP-HPLC on a DNASep® column (Transgenomic, Inc.; San Jose, Calif.) under denaturing conditions. Prior to IP-RP-HPLC, the reaction product was purified using a spin-column containing octadecyl modified, nonporous alkylated poly(styrene-divinylbenzene) beads, as described in U.S. application Ser. No. 09/318,407 and PCT/US00/14956. The spin columns were first incubated with 500 μl of 0.0025M tBuBr (tetrabutylammonium bromide). A volume of 0.0025M tBuBr equal to the reaction volume was added to the reaction mixtures and then loaded onto the column. The columns were then washed twice with 0.0025M tBuBr containing 2 mM EDTA (pH 8.0) . The DNA fragments were then eluted using 70% acetonitrile and load onto the DNAsep® column.

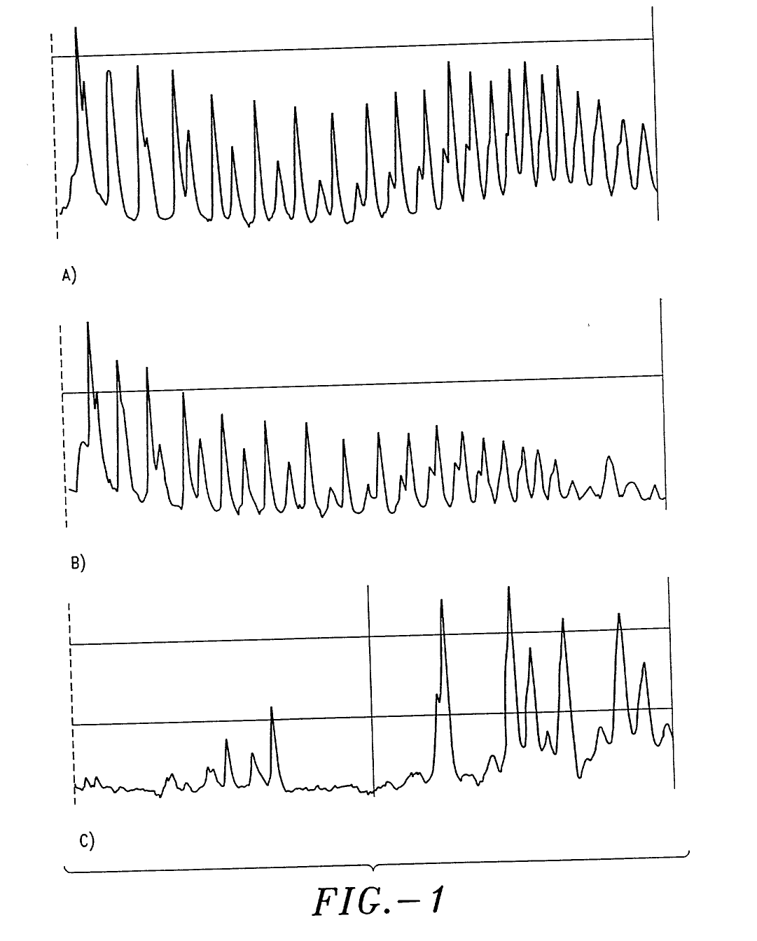

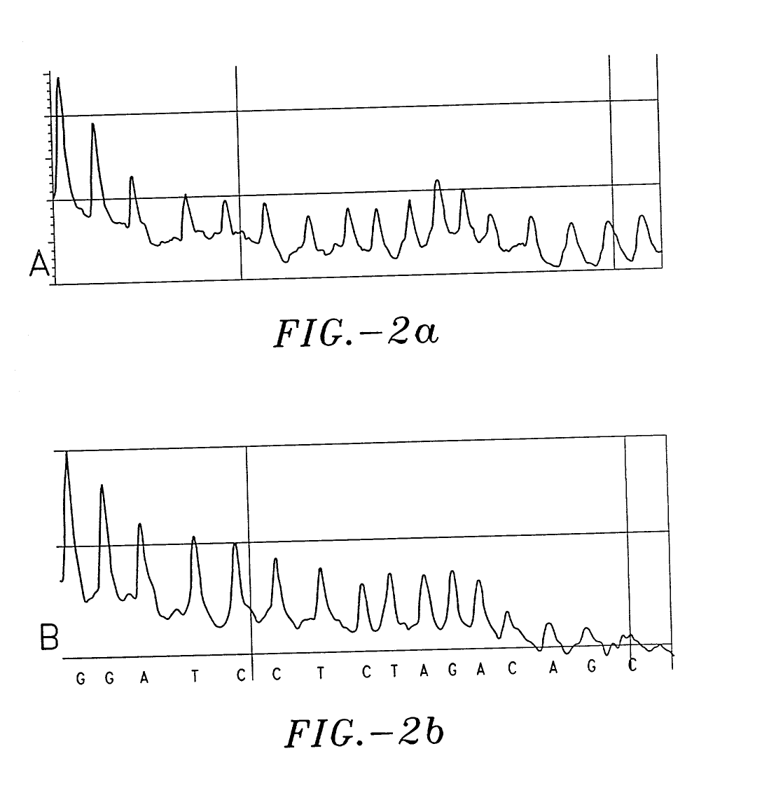

The chromatographic separation was controlled by a WAVE® fragment analysis system (Transgenomic, Inc.; San Jose, Calif.) at 70° C. using fluorescence detection at the appropriate excitation and emission wavelengths (FAM: Ex 494, Em 525; TET: Ex 521, Em 536). The following elution gradient was employed: Buffer A 0.0025 M Tetrabutylammonium bromide (Fisher HPLC), 0.1% acetonitrile, Buffer B 0.0025M, Tetrabutylammonium bromide, 70% acetonitrile. The run was initiated at 30% buffer B, the gradient was extended to 50% buffer B over 12 minutes at a flow rate of 0.9 ml/min, followed by an extension to 60% buffer B over 18 minutes at a flow rate of 0.9 ml/min. The chromatogram (FIG. 1a) shows the effect of hydroxyl radical cleavage of FAM-labeled strand HJ3 in the absence of protein.

The experiment was repeated as above, this time with the inclusion 1 μM E. coli RuvA, a Holliday junction-binding protein. RuvA was purified as described in Sedelnikova et al. (1997) Acta. Cryst. D53:122-24. FIG. 1b shows that the protein protected strand HJ3 from cleavage in the right portion of the chromatogram.

In order to phase the chromatogram, the labeled DNA was used to generate a G+A sequencing ladder by the method of Belikokv and Wieslander (supra). 10 μl of 3% diphenylamine (Aldrich) in formic acid (Aldrich) was added to 75 pmol of the labeled DNA. The reaction volume was then made up to 20 μl with MilliQ water and incubated at room temp for 10 minutes. The reaction was stopped by the addition of 100 μl 0.3M sodium acetate (pH 5.5) and the mixture was extracted three times with water saturated ether. The sample was then placed in a vacuum dryer to remove traces of ether and precipitated by the addition of 3 volumes of ethanol and placed at −20° C. for 30 minutes. The DNA was then precipitated for 15 mins at 15,000 g, re-suspended in Milli Q water (20 μl) and purified by spin-column as described above. 5 μl was then analyzed by IP-RP-HPLC using the conditions described above (FIG. 1c).

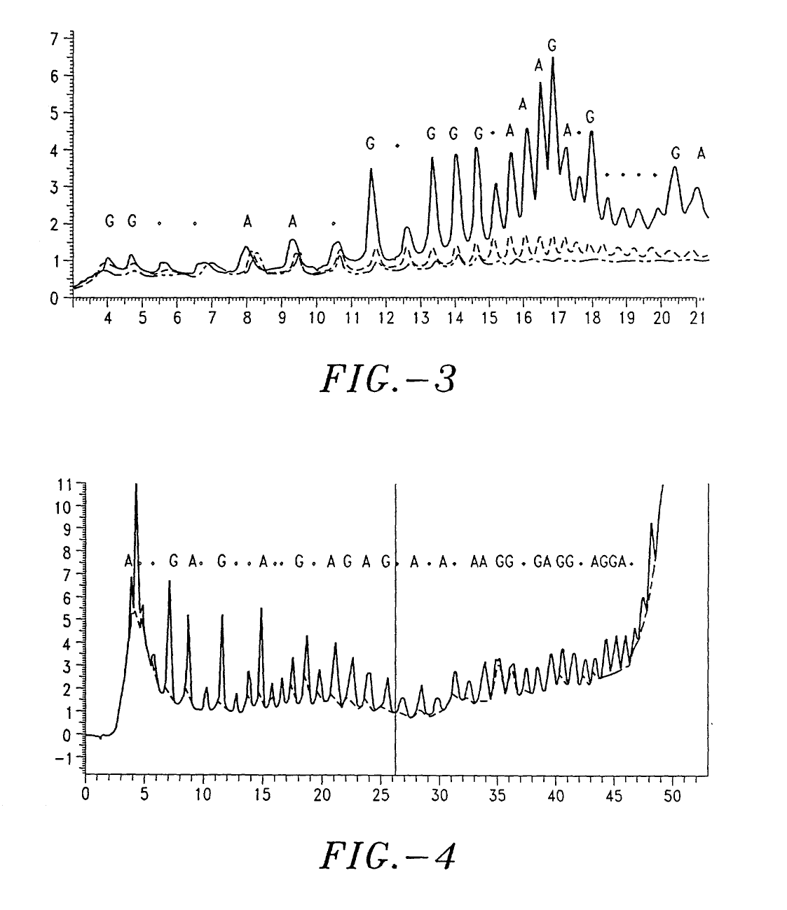

The above procedure was repeated, the only difference being that the TET-labeled HJ4 strand was detected. The resulting chromatograms for the control reaction and the RuvA-including reaction are presented in FIGS. 2a and 2b, respectively.

### EXAMPLE 2

### G+A Sequencing Reactions

This example describes the G+A sequencing of two fluorescently labeled single stranded DNA molecules. The first reaction included 100 pmol of a fluorescently labeled synthetic Holliday junction oligonucleotide (FAM 5′-TGGGTCAACGTGGGCAAAGATGTCCTAGCMTGTMTCGTCTATGACGTT; SEQ ID NO: 5) incubated in 20 μl of 3% diphenylamine (Aldrich) in formic acid (Aldrich) for 5 minutes at room temperature. The reaction was stopped by the addition of 100 μl 0.3M sodium acetate (pH 5.2) and the mixture was extracted three times with water saturated ether. The sample was then placed in a vacuum dryer to remove traces of ether and precipitated by the addition of 3 volumes of ethanol and placed at −20° C. for 30 minutes. The DNA was then precipitated for 15 mins at 15,000 g and re-suspended in Milli Q water (20 μl). 5 μl was then analyzed by IP-RP-HPLC using the conditions described above. The chromatogram is shown as FIG. 3, with the G and A peaks labeled. FIG. 4 shows the chromatogram generated for the labeled oligonucleotide TATA2 (5′ HEX TACCGACGTCATTCGCAGAGCATATMGGTGAGGTAGGATAGCTACGTC; SEQ ID NO: 6) using the same methods and reagents as above.

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein. All references referred to herein, including any patent, patent application or non-patent publication, are hereby incorporated by reference in their entirety.

The invention claimed is:

1. A method for identifying the 3′-end of a polynucleotide appearing as a chromatographic peak in a first IP-RP-HPLC chromatogram, wherein said polynucleotide has been generated by subjecting a precursor nucleic acid of known sequence to a first cleavage reaction, and wherein said polynucleotide shares with said precursor nucleic acid a common 5′-end, the method comprising:
(a) providing a plurality of polynucleotides, wherein said plurality of polynucleotides have been generated by subjecting the precursor nucleic acid of known sequence to a second cleavage reaction, wherein said second cleavage reaction is base-discriminating and is distinct from the first cleavage reaction, and wherein said plurality of polynucleotides share with said precursor nucleic acid and with each other a common 5′-end;
(b) separating said plurality of polynucleotides by IP-RP-HPLC, wherein the IP-RP-HPLC separation conditions are substantially the same as those used to generate said first IP-RP-HPLC chromatogram;
(c) detecting said plurality of polynucleotides as they elute from the IP-RP-HPLC separation, thereby generating a second HPLC chromatogram, wherein the 3′-end of a polynucleotide appearing as a chromatographic peak in said second IP-RP-HPLC chromatogram can be determined based on the known sequence of the precursor nucleic acid; and
(d) comparing said first IP-RP-HPLC chromatogram with said second IP-RP-HPLC chromatogram, wherein the 3′-end of a polynucleotide appearing as a chromatographic peak in said first IP-RP-HPLC chromatogram can be identified based on its elution position relative to a peak appearing in said second IP-RP-HPLC chromatogram whose 3′-end is known.

2. The method of claim 1, wherein said IP-RP-HPLC employs a separation medium that is substantially free of multivalent cations that are capable of interfering with polynucleotide separations.

3. The method of claim 2, wherein said polynucleotide is DNA.

4. The method of claim 3, wherein said separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein said surfaces are non-polar.

5. The method of claim 3, wherein said separation medium comprises polymer beads having an average diameter of 0.5 to 100 microns, said beads being unsubstituted polymer beads or polymer beads substituted with a moiety selected from the group consisting of hydrocarbon having from one to 1,000,000 carbons.

6. The method of claim 5, wherein said beads are substituted with a moiety selected from the group consisting of methyl, ethyl, or hydrocarbon having from 23 to 1,000,000 carbons.

7. The method of claim 3, wherein said separation medium comprises a monolith.

8. The method of claim 3, wherein said separation medium has been subjected to acid wash treatment to remove any residual surface metal contaminants.

9. The method of claim 3, wherein said separation medium has been subjected to treatment with a multivalent cation binding agent.

10. The method of claim 3, wherein said IP-RP-HPLC employs a mobile phase comprising a solvent selected from the group consisting of alcohol, nitrile, dimethylformamide, tetrahydrofuran, ester, ether, and mixtures of one or more thereof.

11. The method of claim 10, wherein said mobile phase comprises acetonitrile.

12. The method of claim 3, wherein said mobile phase comprises a counterion agent selected from the group consisting of lower alkyl primary amine, lower alkyl secondary amine, lower alkyl tertiary amine, lower trialkylammonium salt, quaternary ammonium salt, and mixtures of one or more thereof.

13. The method of claim 12, wherein said counterion agent is selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof.

14. The method of claim 13, wherein said counterion agent is tetrabutylammonium acetate.

15. The method of claim 13, wherein said counterion agent is triethylammonium acetate.

16. The method of claim 12, wherein said counterion agent includes an anion, said anion is selected from the group comprising acetate, carbonate, phosphate, sulfate, nitrate, propionate, formate, chloride, and bromide.

17. The method of claim 3, wherein said detection is achieved using Matched Ion Polynucleotide Chromatography.

18. The method of claim 3, wherein said polynucleotides are detectably labeled.

19. The method of claim 18, wherein said detectable label is fluorescent.

20. The method of claim 19, wherein said detectable label is selected from the group consisting of FAM, JOE, TAMRA, ROX, HEX, TET, Cy3, and Cy5.

21. The method of claim 20, wherein said detectable label is FAM.

22. The method of claim 3, wherein the method is used to identify the 3′-ends of a plurality of polynucleotides appearing as chromatographic peaks in said first IP-RP-HPLC chromatogram by comparing said first IP-RP-HPLC chromatogram and said second IP-RP-HPLC chromatogram.

23. The method of claim 3, wherein said first cleavage reaction involves the use of a DNA cleavage reagent that cleaves DNA that is not protected by a bound protein.

24. The method of claim 3, wherein said cleavage reagent is a hydroxyl radical.

25. The method of claim 3, wherein said cleavage reagent is a nuclease.

26. The method of claim 25, wherein said nuclease is DNase I.

27. The method of claim 23, wherein said protein is a mismatch binding protein.

28. The method of claim 27, wherein said mismatch binding protein is selected from the group consisting of T4 endonuclease VII, T7 endonuclease I, S1 nuclease, mung bean endonuclease, MutY protein, MutS protein, MutH protein, MutL protein, cleavase, and CELI.

29. The method of claim 28, wherein said mismatch binding protein is CELI.

30. The method of claim 23, wherein said protein is a protein involved in DNA transcription, replication, and recombination.

31. The method of claim 23, wherein said protein is selected from the group consisting of transcription factors, enhancers and repressors.

32. The method of claim 23, wherein said protein binds to a Holliday junction.

33. The method of claim 24, wherein said protein is RuvA.

34. The method of claim 3, wherein said second cleavage reaction is a reaction that results in the specific cleavage of a DNA sequence preferentially after one, two or three of the bases selected from the group consisting of adenine, guanine, cytosine, and thymine.

35. The method of claim 34, wherein said second cleavage reaction is a chemical cleavage DNA sequencing reaction.

36. The method of claim 35, wherein said sequencing reaction is an A+G specific DNA sequencing reaction.

37. The method of claim 36, wherein said DNA sequencing reaction is based on partial acidic hydrolyses of DNA in the presence of diphenylamine and proceeds via depurination/5′,3′-elimination.
